(19) United States
(12) Patent Application Publication
(10) Pub. No.: US 2005/0032133 A1
(43) Pub. Date: Feb. 10, 2005

(54) METHOD OF DETERMINING WHETHER A PATIENT AFFLICTED WITH CANCER CAN BE TREATED BY A THERAPEUTIC AGENT THAT TARGETS THE 17-1A ANTIGEN

(76) Inventors: PAULA LOUISE BARRATT, STEVENAGE (GB); JAMES SCOTT CROWE, STEVENAGE (GB); JONATHAN HENRY ELLIS, STEVENAGE (GB); NICHOLAS TIMOTHY RAPSON, STEVENAGE (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/494,169

(22) PCT Filed: Nov. 5, 2002
(86) PCT No.: PCT/GB02/05027

(30) Foreign Application Priority Data
Nov. 5, 2001 (GB) 0126531.3

(51) Int. Cl. G01N 33/574

(52) U.S. Cl. 435/7.23

(57) ABSTRACT

The present invention provides methods for identifying human cancer patients whose cancerous cells overexpress the 17-1A antigen to such an extent as determined herein that the patients may be considered as suitable for treatment with a therapeutic 17-1A antigen binding agent such as an antibody. Preferably the therapeutic antibody is Edrecolomab. Kits for use in such methods and methods of treating patients so identified are also provided.

### FIELD OF THE INVENTION

The present invention concerns methods for identifying individual human patients afflicted with colon cancer who are more likely to benefit from treatment that targets the 17-1A antigen than the general colon cancer-afflicted patient population as a whole. Methods of medical treatment, particularly of those patients suspected of being afflicted with disseminated/occult cancer cells following surgery to remove the primary tumour, predictive kits, and treatment kits are also provided. Other aspects, objects and advantages of the present invention will be apparent from the description below.

### BACKGROUND OF THE INVENTION

Cancer of the large bowel is the second or third most common cause of cancer-related death in Europe and the USA. The incidence of colorectal cancer varies greatly world-wide, being much lower in less developed countries such as India (1-2 per 100,000 per annum) compared to the USA and the UK (1545 per 100,000 per annum) (Parkin et al. 1992).

Colon and rectal cancer are commonly defined by Dukes' stage which encompasses depth of invasion into the bowel wall, lymph node involvement and distant metastases. Dukes' stage A carcinomas invade the bowel wall, but have not spread beyond the muscularis propria, Dukes' stage B carcinomas invade beyond the muscularis propria, but lymph nodes are not involved, Dukes' stage C carcinomas involve the lymph nodes. Subsequently stage D was introduced to classify patients with hepatic metastases (Dukes 1932; Turnbull et al. 1967).

Dukes' stage D tumours are treated with surgery, where possible, to remove or debulk tumour, followed by chemotherapy to reduce rate of relapse or disease progression. Current chemotherapy regimes are typically structured around treatment with 5-fluorouracil (5FU). Earlier stage disease is treated with surgery which is potentially curative. However relapse can be caused by occult metastases present at the time of surgery, dispersal of cells (micrometastases) at the time of surgery or local residual disease. Metastatic spread occurs predominantly in the liver. If it occurs within two. years of resection of the primary tumour It is believed that in the majority of these cases the metastases were already present at the time of surgery, although occult to preoperative investigation (Finlay & McArdle, 1983). It has been demonstrated that tumour cells can be detected in the portal circulation at the time of surgery (Fisher & Turnbull, 1955) and this may be the cause of liver metastases developing around or more than 2 years after ‘curative’ surgery. Most tumour cells shed into the venous system are destroyed, but some can survive to form metastases.

As apparently curatively resected patients frequently suffer relapse, adjuvant treatment is often given, especially for Dukes' stage C disease which is considered the highest risk. The treatment given is usually 5FU-based chemotherapy. It produces modest survival improvements but is not effective in all patients, one reason being the preponderance of dormancy in metastatic tumour cells. For this reason alternative adjuvant treatments which can eradicate dormant tumour cells could have an important role in colorectal cancer therapy.

5FU based chemotherapy is frequently associated with toxicity including leucopenia, nausea, vomiting, diarrhoea, stomatitis and in rare cases it is fatal. As chemotherapy is effective to differing degrees in individual patients and has significant adverse effects, it would be ideal to identify those patients for whom it would give most benefit, in which case the overall benefit may outweigh the toxicity. It would also be possible to identify those patients to whom it gives little or no benefit so that unnecessary toxicity can be avoided and, if available, alternative therapies used. This would represent a significant improvement in patient care.

Edrecolomab (Panorex™ also Adjuqual™) is an IgG2a murine monoclonal antibody directed to the human epithelial cell molecule known as 17-1A antigen.

The molecule is also known by alternative names such as EpCAM, EGP40, GA733-2, KSA, 40 kD antigen and ESA. It is expressed on several epithelial tumour types as well as normal epithelial tissue, and is thought to be involved in cell adhesion processes (Litvinov et al. J Cell Biol, 1994, 125, 437446; Litvinov et al. Cell Adhesion and Communication, 1994, 2, 417-428, the entire contents of which are incorporated herein by reference and to which the reader is specifically referred).

Edrecolomab is also known in the literature as Mab CO17-1A, Mab 17-1A and Panorex™/Adjuqual™. Treatment with edrecolomab has been reported to demonstrate significant efficacy in a study of 189 Dukes' stage C colorectal cancer patients. In this study patients received surgery to remove the primary tumour followed by randomisation to an observation regimen or to treatment with edrecolomab (an initial infusion of 500 mg followed by monthly doses of 100 mg for 4 months). Seven years of follow up data demonstrated that treatment reduced overall mortality by 32% (p<0.01) and recurrence by 23% (p<0.04). There was a significant improvement in overall survival (p<0.01) and disease-free survival (p=0.02). It appears that the survival benefit of edrecolomab was due to prevention of the development of distant metastases as there was no influence of treatment on time to local relapse (Riethmuller et al. 1998). This reduction in mortality was equivalent to that previously seen in response to 5FU based chemotherapy treatment (Moertel et al. 1995).

Edrecolomab is thought to mediate eradication of tumour cells principally via antibody dependent cellular cytotoxicity (ADCC). Other effector functions of antibodies may also be involved in its mechanism of action. These might include opsonisation, the activation of complement components or the induction of an anti-idiotype cascade. Edrecolomab is thought to be most effective against single cells and small cell aggregates rather than established tumour masses because of accessibility of the antibody and effector cells to the tumour cells. This would explain the ability of edrecolomab to reduce time to distant metastases, but not local recurrence in the Riethmuller study. Edrecolomab is also expected to be active against dormant tumour cells, as immunological effector mechanisms do not depend on tumour cells entering the cell cycle.

There have been some investigations into the prognostic value of 17-1A antigen expression but not in predicting reponse to 17-1A antigen targeting therapy.. Studies in lung and prostate cancer have found that the 17-1A antigen does not have prognostic value, even though for prostate cancer its expression level was seen to increase in the transformation from normal to tumour (Poczatek 1999) and for lung cancer its expression increased with disease progression (Piyathilake 2000). A study of breast cancer patients found that overexpression of 17-1A correlated to decreased disease-free and overall survival (Gastl 2000). From the current literature it is clear that the prognostic significance of the level of expression of 17-1A antigen has not been determined for cancer of the colon.

However, in contrast to the general prognostic significance of 17-1A antigen expression different results may be obtained when considering the predictive value of 17-1A antigen expression for response to 17-1A antigen targeted therapy.

It is appreciated that complete absence of 17-1A antigen on tumour cells precludes benefit from a 17-1A antigen targeting therapy. However, currently there is no notion of what level of expression is required for sufficient benefit from a 17-1A antigen targeting therapy. As such treatment with anti-17-1A antigen targeting therapies where some expression of 17-1A antigen in the tumour is suspected regardless of the level of expression of the 17-1A antigen is sufficient to allow benefit from treatment with a 17-1 A targeting therapy (Punt CJA et al, The Lancet, 360, 671-677, 2002, Riethmuller et al 1994). The lack of any method to identify those patients which benefit particularly from said therapy is corrected by the present invention and makes possible a rational patient selection strategy.

As used herein by “prognostic value” and the like we mean indicating the risk of disease progression or death independently of whether or which therapy is given. In contrast, a “predictive factor” and the like, as used herein, indicates to what extent a particular therapy gives benefit (Hayes D F, 1998, Breast Cancer Res Treat, 52,305-319).

It is an object of the present invention to provide a method of identifying those individual human patients likely to benefit most from therapies targeted to the 17-1A antigen. Typically these will be medicaments that specifically bind the 17-1A antigen expressed on the cancerous cell leading to apoptosis and/or necrosis or otherwise subject the cancerous cells to a response from the patients immune system resulting in erradication. Examples of specific 17-1A antigen binding medicaments are Edrecolomab and MT201 (Naundorf S.et al 2002 Int. J. Can. 100, 101-110).

The present inventors have found that the level of expression of the 17-1A antigen on colon cancer cells is predictive of the response that a human patient afflicted as such will have to a treatment that targets the 17-1A antigen. The present invention therefore provides means for identifying those human patients that express the 17-1A antigen at a level that encourages clinical benefit from the treatment of a 17-1A targeted treatment, e.g. an anti-17-1A immunoglobulin.

The present invention furthermore provides methods for identifying human cancer patients whose cancerous cells overexpress the 17-1A anitgen to such an extent as determined herein that the patients may be considered as suitable for treatment with a therapeutic 17-1A antigen binding agent such as an antibody. Preferably the therapeutic antibody is Edrecolomab or MT201. Kits for use in such methods and methods of treating patients so identified are also provided.

All prior publications referred to herein are expresely and entirely incorporated herein by reference.

### SUMMARY OF THE INVENTION

In accordance with the present invention there is provided a method for determining the suitability of a human patient afflicted with colon cancer or suspected of being afflicted with disseminated and/or occult cancer cells for treatment with a therapeutic agent that targets (e.g. specifically binds) the 17-1A antigen expressed by said cancer or cancer cells which method comprises the steps of;
(a) obtaining an ex vivo sample (e.g. a portion of the resected primary tumour or a biopsy of primary/secondary or metastatic tumour and/or a blood or bone marrow sample where detection of disseminated cancer cells is desired) of the cancerous colon cells from said patient;
(b) assessing (e.g. by measuring) the expression of 17-1A by said cells;
(c) comparing the expression of step (b) with a reference expression level;
(d) determining whether the comparison made in step (c) is indicative of said cancerous cells overexpressing 17-1A (e.g. 17-1A antigen) for example, determining that said cells have a higher expression of 17-1A than the reference expression level wherein overexpression is determinative of suitability;
(e) optionally administrating to a human patient identified in step (d) as being suitable an agent that targets the 17-1A antigen.

A method of treating a human patient afflicted with colon cancer or suspected of being afflicted with occult cancer cells, particularly following surgery to remove a tumour, which patient has been identified as suitable by the method disclosed supra which method comprises the step of treating said patient with a therapeutic agent that targets the 17-1A antigen is also provided.

A method of treating a human patient suspected of being afflicted with occult cancer cells, particularly those derived from a colon cancer tumour or otherwise responsive to 17-1A antigen targeted treatment which method comprises the step of identifying whether said patient is suitable for treatment with a therapeutic agent that targets the 17-1A antigen according to the method described supra is also provided.

A kit comprising an agent that is capable of binding to 17-1A (preferably 17-1A antigen) which agent comprises a detectable moiety capable of being detected when bound to said 17-1A together with instructions for performing the method of identifying a human patient as described supra optionally together with instructions for treating a human patient so identified is also provided.

According to another aspect of the invention, the methods and kits of the invention may be used in combination with other predictive methods for the prediction of benefit from other therapies not directed at 17-1A antigen. In preferred embodiments, these other therapies are used in combination with 17-1A antigen targeted therapy.

### DETAILED DESCRIPTION OF THE INVENTION

The term “17-1A” is intended to refer to all aspects of 17-1A expression. It therefore includes genomic DNA, cDNA, mRNA and the 17-1A protein (herein referred to as “17-1A” antigen).

It will be understood that the term “suitable for treatment” and grammatical variations thereof refers only to whether the patient is more likely to benefit from treatment that targets the 17-1A antigen. There may of course be other considerations that dictate whether the patient actually undergoes such treatment, these considerations being within the purview of the attending physician.

It will also be understood that the identification of a patient suitable for treatment according to the present invention does not imply that in all cases the treatment will be a success (by which we mean at least some clinical benefit). The present invention facilitates the identification of those individual human patients afflicted with colon cancer, disseminated cancer cells or suspected of being afflicted with occult cancer cells overexpressing the 17-1A antigen who are more likely to benefit from treatment that targets (preferably specifically targets) the 17-1A antigen than the general human colorectal cancer afflicted patient population as a whole. Of course other factors such as age and the general health status of the patient may influence the degree of clinical benefit such as final outcome.

By “overexpressing” we mean that the cancerous cells are expressing 17-1A antigen to a sufficiently high degree that the human patient may be considered as suitable for treatment as defined supra.

In the first instance a sample of cancerous colon cells is obtained either directly from the tumour itself or more typically from the resected primary tumour following surgery. The sample size needed to perform the methods of the present invention is of course ultimately limited by the amount of tumour available from the patient. However, generally speaking the size of sample required will be that typically used in standard immunohistochemistry techniques and tissue microarray histochemistry.

Determining the expression of 17-1A may be undertaken qualitatively, quantitatively, or semi-quantitatively.

Preferably, 17-1A antigen expression levels are determined by detecting expression levels of the antigen itself. Alternatively, but less preferred, determination may be achieved indirectly through measurement of various aspects of 17-1A expression such as 17-1A gene copy number, cDNA or mRNA. Such indirect methods of course require a determination of the degree of correlation between its expression and expression of the 17-1A antigen.

The expression level of 17-1A DNA,cDNA, mRNA etc may be determined by methods known or apparent to those skilled in the art. For example, levels may be determined by measuring the degree of hybridisation under stringent conditions of an oligonucleotide whose base sequence is complementary to a region of the 17-1A gene (copy number)/cDNA/mRNA that is specific for 17-1A, so that the hybridisation that does occur can be attributed to 17-1A expression. An example of this approach is fluorescence in situ hybridization (FISH). The exact length and composition of the complementary oligonucleotide necessary to achieve these ends can be readily determined by the skilled person however, it will generally not be less than 8 nucleotides and will typically be between 8 and about 50 nucleotides in length. The complementary oligonucleotide is contacted with a sample of cell lysate or a tissue section containing the 17-1A DNA or RNA and left for a sufficiently long length of time to allow hybridisation to occur. Stringent hybridisation conditions may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Alternatively, mRNA levels may be amplified according to methods well known or apparent to those skilled in the art such as a reverse transcriptase polymerase chain reaction (RT-PCR) e.g. Taqman™. The RT-PCR method maybe quantitative.

The sequence of 17-1A gene exons and flanking regions, mRNA and antigen can be found in Linnenbach, A J., Seng, B A., Wu, S., Robbins, S., Scollon, M., Pyrc, J J., Druck, T., Huebner, K. (1993) Retroposition in a family of carcinoma associated antigen genes. Mol Cell Biol 13. 1507, the entire contents of which are incorporated herein by reference and to which the reader is specifically referred. The entire 17-1A genomic sequence can be found by searching a human genome database such as Genebank with the 17-1A mRNA sequence registered under accession number AH003574. Preferably associated with the complementary oligonucleotide is detectable moiety, for example a radiolabel, whose detection is measurable. Southern and Northern Blotting may also be employed for the detection of DNA and RNA respectively.

Preferably however, levels of expression of the 17-1A antigen are assessed e.g. measured. Again, there are a number of methods available to those skilled in the art. For example, Western blotting may be employed according to methods well known and routinely used. Preferably, immunohistochemistry is used whereby the 17-1A antigen is detected and whose expression on the cancerous cells is measured using a 17-1A specific binding agent, for example an anti-17-1A antibody with a detectable moiety for example coupled to a radiolabel or fluorescence label or an enzyme of use in a colormetric reaction. This antibody may bind the 17-1A antigen directly or alternatively may bind another antibody which itself binds the 17-1A antigen. The anti-17-1A antibody may be directed against the 17-1A protein sequence and/or its glycosylation. Techniques such as these are standard within the field of immunohistochemistry. Generally speaking, a sample of cancerous cells is typically obtained from a resected primary tumour, and formalin fixed and embedded in paraffin wax. The 17-1A antibody is then incubated with the cells and the degree of binding of the 17-1A antibody to the cell sample is determined according to standard techniques of the art. Typically sections of tissue are cut from a formalin-fixed and paraffin-embedded sample using a microtome. They are collected on a glass slide, dried, de-waxed by placing in xylene and rehydrated by passage through graded alcohol to water. An antigen retrieval method may be applied, possibly using a solution of trypsin or a citrate buffer and possibly in conjunction with microwaving or high pressure. After a washing step endogenous molecules, such as peroxidase and biotin, may be blocked. Tissue sections are then exposed to a primary antibody which is typically specific for the antigen being detected. After washing tissue sections are usually exposed to a secondary antibody which specifically binds to the primary antibody and can be detected using a variety of detection methods; frequently these are peroxidase based and/or alkaline phosphatase based and result in a colorimetric change at the sites where primary antibody bound to the cells. Sections can then be stained with haemotoxylin or another nuclear or cytoplasmic dye to facilitate examination of the tissue. Staining and degree of staining can then be assessed using a light microscope or by computer based image analysis.

However, merely being able to conduct immunohistochemistry for 17-1A antigen does not enable a suitable patient population to be selected for treatment. For example, when the anti-17-1A antibody illustrated herein (3622W94) is used at a concentration of 2.8 μg/ml, more than 99% of tumour samples tested show binding. By the term “showing binding” and grammatical variations thereof we mean that binding of the antibody is visually detectable to the human eye at at least four times magnification, preferably at least 20 or 40 times magnification.

In contrast to this uniform statning, the present inventors have found that when appropriate immunohistochemical conditions are employed, it is possible to identify a specific subgroup of human patients for whom a therapeutic agent targeting the 17-1A antigen is especially beneficial in its clinical effect. For example, when a batch of detection antibody 3622W94 described herein is employed in immunohistochemistry for the 17-1A antigen at a concentration of 2.8 ng/ml or thereabout, it is possible to rank the human colon cancer population into a group comprising 44% of the total population as suitable for treatment; and another group comprising 56% of the population for whom such treatment is less suitable. It will be apparent that particular aspects of the immunohistochemistry method and conditions employed contributed to the determination of the useful concentration of 17-1A detection antibody. For example, antigen retrieval conditions selected, the detection/visualisation method chosen (e.g. machine executed image analysis), activity/affinity of the particular batches of reagent used in the experiment. Accordingly modification of the particular methods exemplified herein may lead to a modification of the useful concentration of detection antibody/reagent employed. Generally speaking though, the useful concentration will fall with a range of approximately 1.4 ng/ml to 14 ng/ml.

It will be apparent that a concentration of exactly 2.8 ng/ml for 3622W94 antibody or other antibody used to detect the 17-1A antigen is not absolutely necessary to practice the invention. The value of 2.8 ng/ml refers to 3622W94, moreover a particular batch of 3622W94. Account needs to be taken of the fact that batch to batch variation occurs and appropriate recalibration of the staining method to take into account of this needs to be carried out. Furthermore, methods for determining the necessary concentration for an anti-171A antibody other than 3622W94, in order to provide a similar result as 3622W94 equally necessitates recalibration of the staining method. Methods for achieving these are provided in Example 10 below.

Thus there is also provided a method for identifying a human patient afflicted with colorectal cancer and/or suspected of being afflicted with occult cancer cells as being suitable for treatment with a 17-1A antigen binding agent, which method comprises the steps of;
(a) obtaining an ex vivo sample of a colon tumour or bodily tissue or fluid suspected of containing occult cancer cells;
(b) contacting said sample of step (a) with a solution of 3622W94 antibody at a concentration of antibody of 2.8 ng/ml or thereabout under conditions favourable for binding;
(c) detecting binding of the antibody of step (b), wherein binding of the antibody is indicative of said patient being suitable.
(d) Optionally treating a patient identified in step (c) with a therapeutic agent which binds to the 171A antigen.

Step (b) of the above described method may alternatively use an anti-17-1A antibody other than 3622W94 at a concentration providing substantially the same degree of binding as 3622W94 under the same conditions. That is, the concentration of 17-1A antibody required to give the same degree of binding as 3622W94 may be achieved simply by titrating the concentration of the 17-1A antibody until an identical or similar degree of binding is achieved as that observed with 3622W94.

The therapeutic agent is preferably an anti-17-1A immunoglobulin such as an 17-1A antibody and may be derived from animal plasma and be polyclonal but is preferably monoclonal and/or recombinant. The term “antibody” includes humanised/CDR-grafted/chimeric or otherwise engineered antibodies and includes fragments such as Fab, F(ab), Fv, F(ab′)₂, single chain antibody molecules and multispecific antibodies such as bispecific and diabodies. Most preferably the antibody is Edrecolomab or MT201

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG I, IgG2, IgG3, IgG4, IgA1 and IgA2. The heavy-chain constant domains that correspond to the different classes of immunoglobulins, IgA, IgD, IgE, IgG, and IgM, are called α, β, ε, γ, and μ respectively. The subunit structures and three-dimensional configurations of different classes of immunoglobulins are well known. Antibodies of the present invention may be of any appropriate class/subclass, but preferably have the ability to recruit effector cells/substances, e.g. can mediate antibody dependent cellular cytotoxicity (ADCC) or complement mediated lysis (CML). They may also have the ability to be taken up by antigen-presenting cells to induce an anti-idiotype response of therapeutic effect. Alternatively antibodies of the present invention may be conjugated to cellular toxins whereby effector functions are not required for therapeutic effect.

There is also provided a kit for use in the methods described herein comprising a 17-1A immunoglobulin e.g. antibody having coupled thereto a detectable moiety together with instructions for measuring 17-1A antigen levels at a dilution/concentration that enables a distinction to be made between those cancerous cells over-expressing 17-1A antigen and those that are not. Optionally the kit may also comprise a reference standard of 17-1A expression. The standard may take the form of a visual reference card wherein the user of the kit can visually compare the binding obtained with the patient sample against a standard of binding indicative of either normal 17-1A antigen expression and/or over-expression. Alternatively the standard may take the form of a control sample of cells having a pre-determined level of 17-1A expression which are provided with the kit and processed alongside the patient sample. The binding is then compared between the control and the patient sample to determine 17-1A antigen over-expression. In a preferred embodiment, the control sample of cells are obtained from non-cancerous tissue of the patient itself to provide an internal control. Preferably they are obtained from the same tissue type as the cancerous cells, e.g. colon tissue. As will be apparent to those skilled in the art, embodiments of the kit will be adapted according to the method used to assess 17-1A antigen binding (e.g. visually with the human eye or machine executed analysis).

A treatment kit comprising a predictive kit as described supra together with a therapeutically effective amount of a therapeutic agent that targets (e.g. specifically binds) upon administration cells overexpressing the 17-1A antigen, particularly colon cancer cells is also contemplated. An example of a suitable therapeutic is an anti-17-1A immunoglobulin.

The 17-1A targeted treatment of a patient identified herein maybe combined with other therapeutic agents in a method of treating a patient afflicted with cancer. The other therapeutic agent may be administered simultaneously, sequentially or separately with the 17-1A targeted treatment and examples of such other therapeutic agents include established chemotherapeutic agents such as 5FU.

The present invention may be applied to the treatment of other tumour types. The tumour types which may be treated with a 17-1A targeted treatment such as a 17-1A antibody optionally in combination with another therapeutic agent are those of any origin that express the 17-1A antigen. For example colorectal, breast, gastric, oesophageal, prostate, lung, pancreatic and ovarian cancer. The kit and treatment kit as hereinbefore described may be used to measure 17-1A, particularly 17-1A antigen in these additional cancers.

In accordance with the present invention there is provided a method of treating a human patient suspected of being afflicted with occult cancer cells, which method comprises the steps of;
(a) removing a tumour or portion thereof from a human patient;
(b) determining the level of 17-1A expression of the tumour or portion thereof of step (a);
(c) comparing the level of 17-1A expression of step (b) with a reference level expression of 17-1A;
(d) determining whether the comparison of step (c) is indicative of over-expression of 17-1A wherein overexpression is indicative of suitability for a treatment that targets the 17-1A antigen;
(e) administrating to the human patient if determined to be suitable in step (d) a therapeutic agent that targets the 17-1A antigen optionally in combination with another therapeutic agent.

### EXEMPLIFICATION

The present invention is now described by way of example only and with reference to the following figures in which:

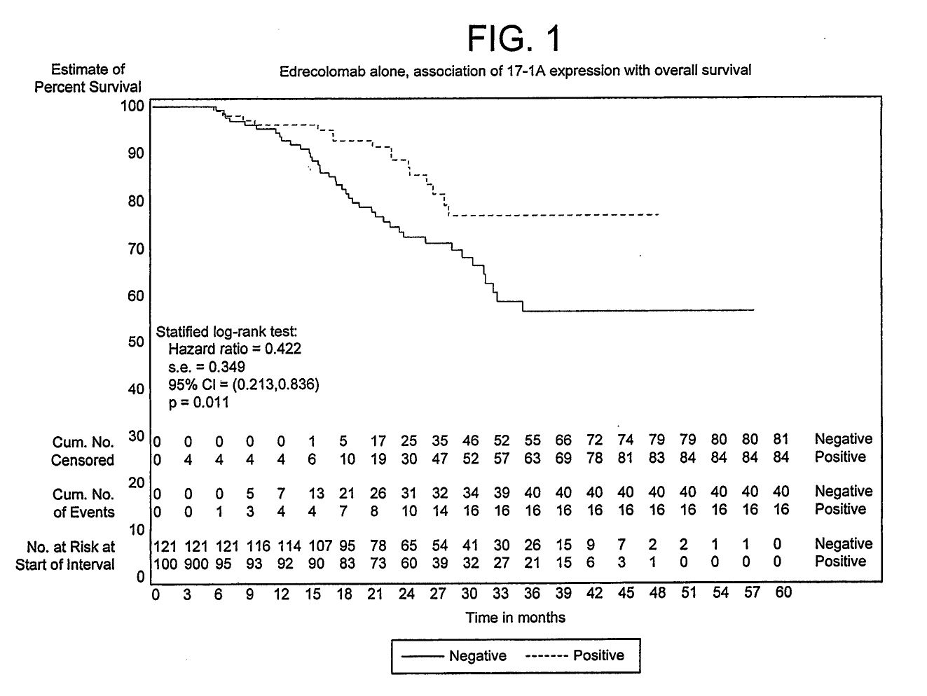

FIG. 1. This is a Kaplan-Meier overall survival plot comparing the survival of 17-1A positive and negative patients overtime. Hazard ratio (HR) indicates the risk of death associated with being 17-1A positive or 17-1A negative. A hazard ratio of 1 indicates no difference in risk of death between the 2 groups while a HR<1 indicates a lesser risk of event for patients positive for 17-1A. In this case the p value is <0.05 indicating that the HR is significant, i.e. the difference in risk of death between the 17-1A positive and the 17-1A negative patients is significant. These patients were all treated with edrecolomab monotherapy. Expression of the 17-1A antigen was detected using FITC-3622W94 at a concentration of 2.8 ng/ml. “s.e.” is standard error of the HR, “95% Cl” is 95% confidence interval. “Cum. No. censored” is the cumulative number of patients censored, where ‘censored’ means removed from the curve. Patients are censored from the last point in time at which relevant clinical information was collected onwards. “Cum. No. of events” is cumulative number of events, where an event means death. “No. at risk at start of interval” is the number of patients represented on the graph at each time point and it reduces as patients have an event or are censored.

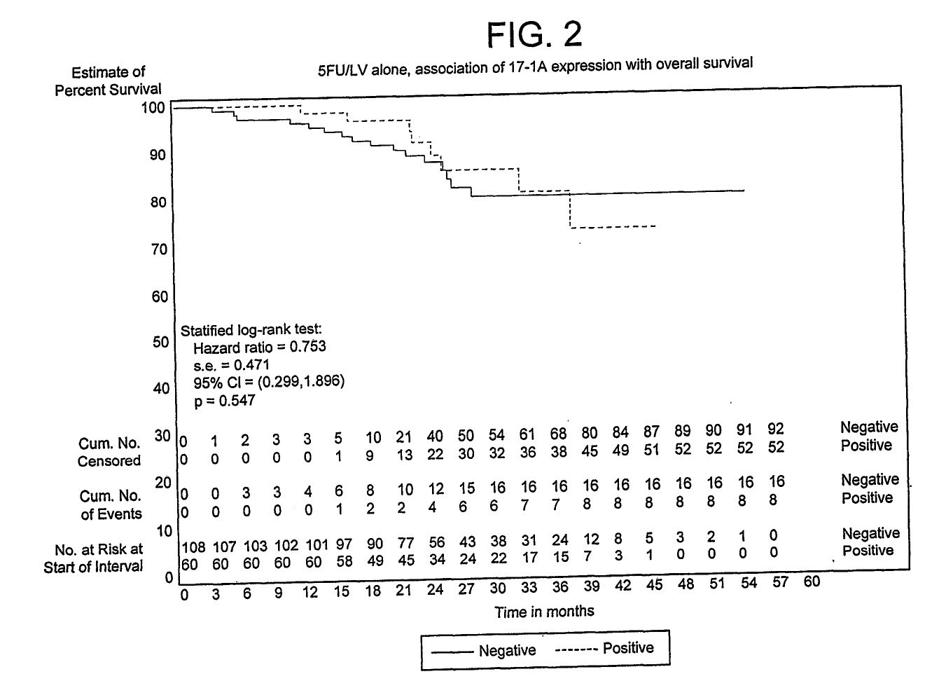

FIG. 2. Overall survival in patients treated with 5FU/LV alone—comparison of 17-1A positive and negative patients (2.8 ng/ml 3622W94 antibody dilution). A HR<1 indicates a lesser risk of event for patients positive for 17-1A.

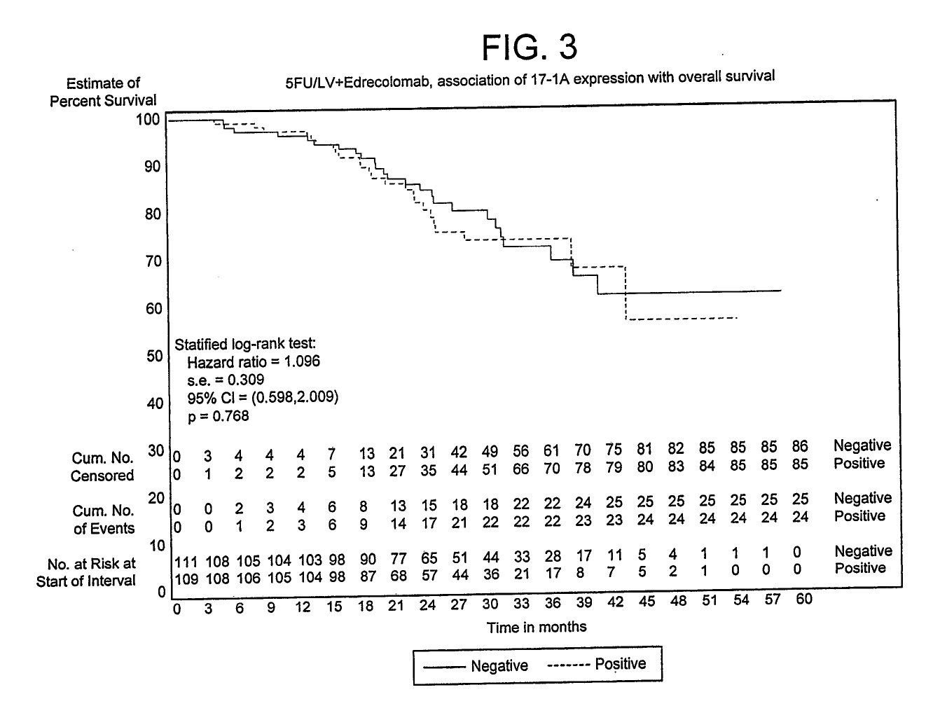

FIG. 3. Overall survival in patients treated with 5FU/LV+edrecolomab—comparison of 17-1A positive and negative patients (2.8 ng/ml 3622W94 antibody dilution). A HR>1 indicates a greater risk of event for patients positive for 17-1A.

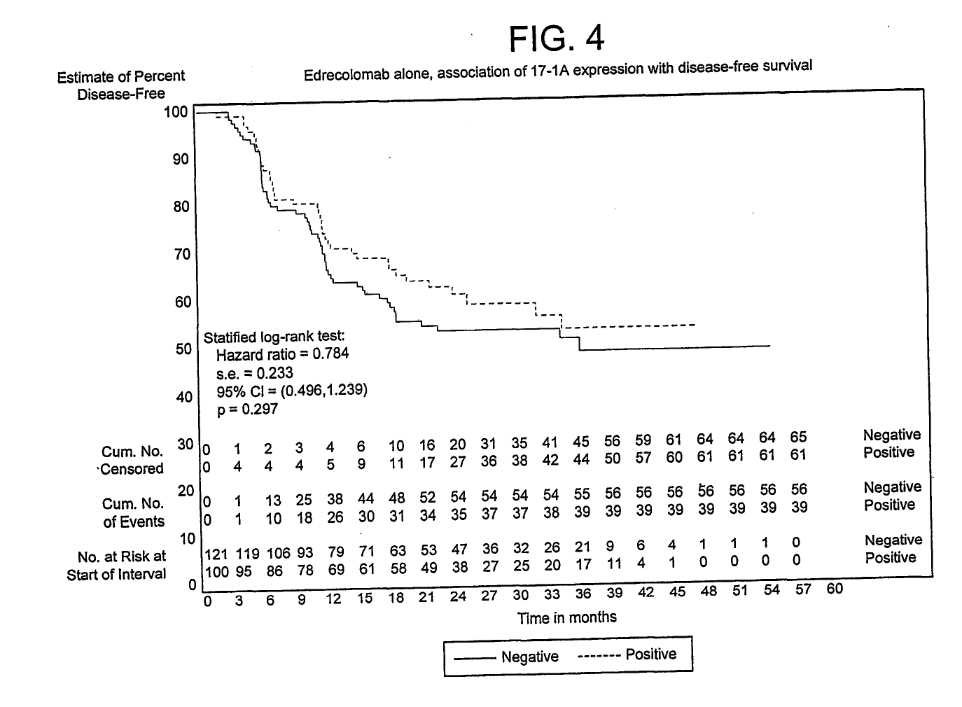

FIG. 4. This is a Kaplan-Meier disease-free survival plot comparing the disease-free survival of 17-1A positive and negative patients over time. Patients are treated with edrecolomab alone. Detection of the 17-1A antigen was with FITC-3622W94 at a concentration of 2.8 ng/ml. A HR<1 indicates a lesser risk of event for patients positive for 17-1A. For a disease-free survival plot ‘event’ means disease recurrence or death.

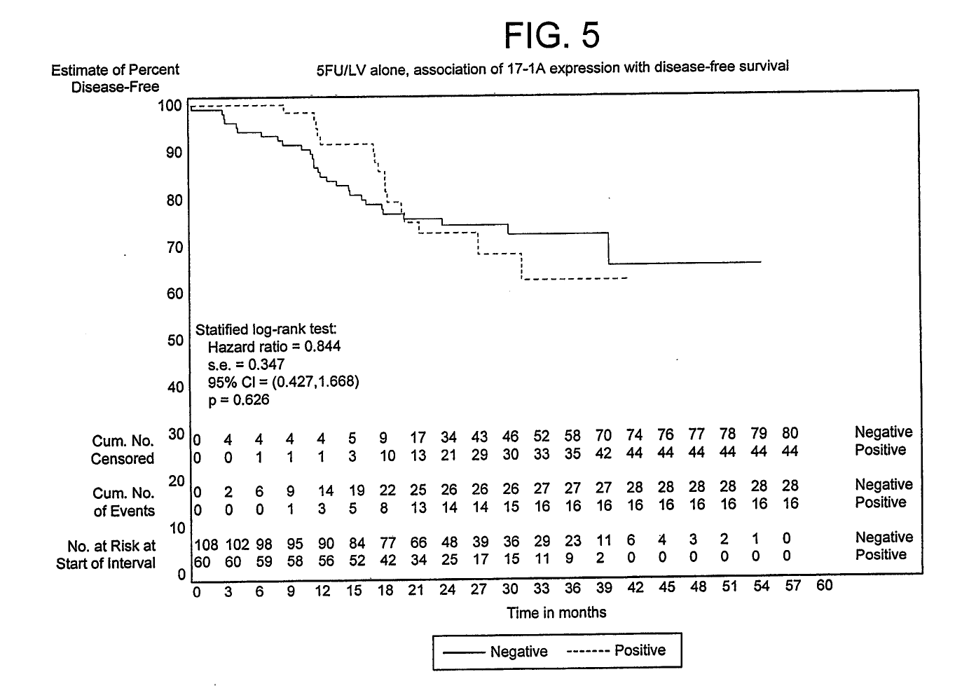

FIG. 5. Disease-free survival in patients treated with 5FU/LV alone—comparison of 17-1A positive and negative patients (2.8 ng/ml 3622W94 antibody dilution). A HR<1 indicates a lesser risk of event for patients positive for 17-1A.

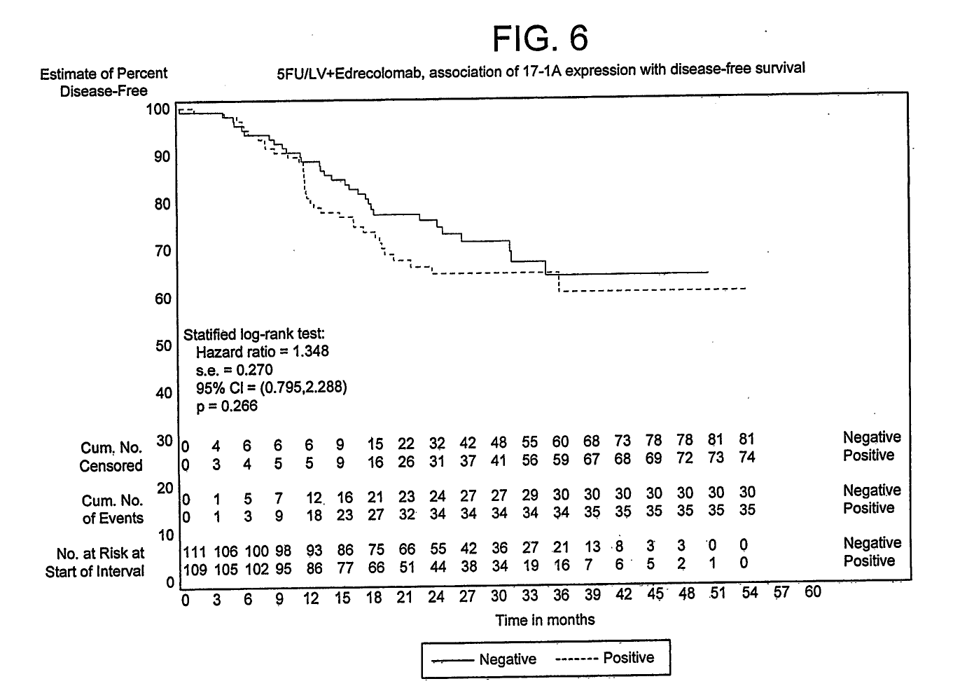

FIG. 6. Disease-free survival in patients treated with 5FU/LV+edrecolomab—comparison of 17-1A positive and negative patients (2.8 ng/ml 3622W94 antibody dilution). A HR>1 indicates a greater risk of event for patients positive for 17-1A.

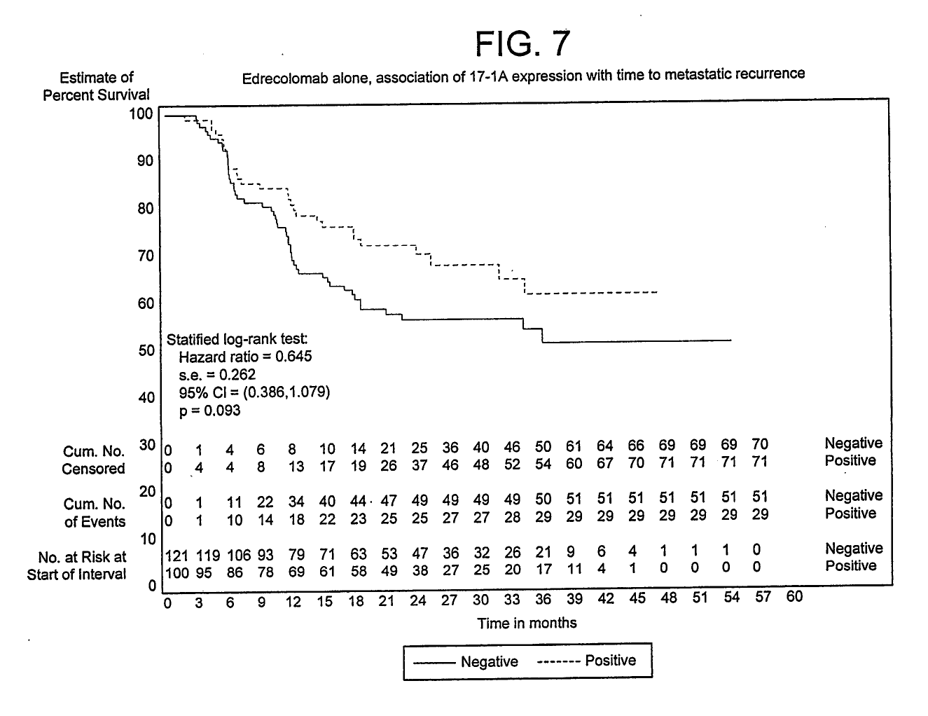

FIG. 7. Time to metastatic recurrence in patients treated with edrecolomab alone—comparison of 17-1A positive and negative patients (2.8 ng/ml FITC-3622W94). A HR<1 indicates a lesser risk of event for patients positive for 17-1A. ‘event’ means a metastatic recurrence, i.e. not local or death.

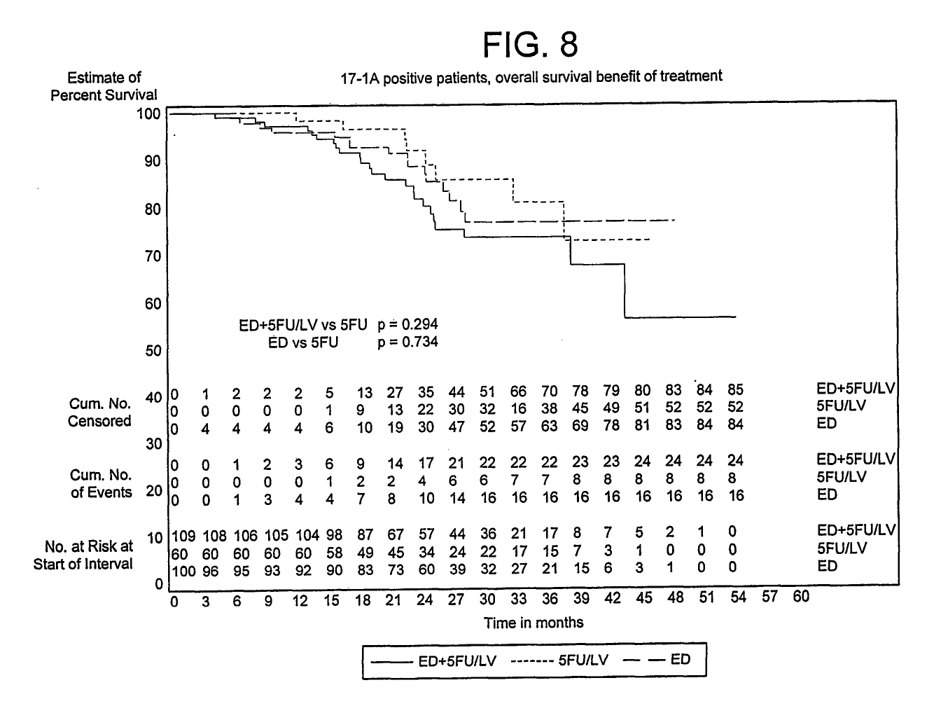

FIG. 8. Overall survival of 17-1A positive patients (2.8 ng/ml 3622W94 antibody dilution)—comparison of the treatment arms.

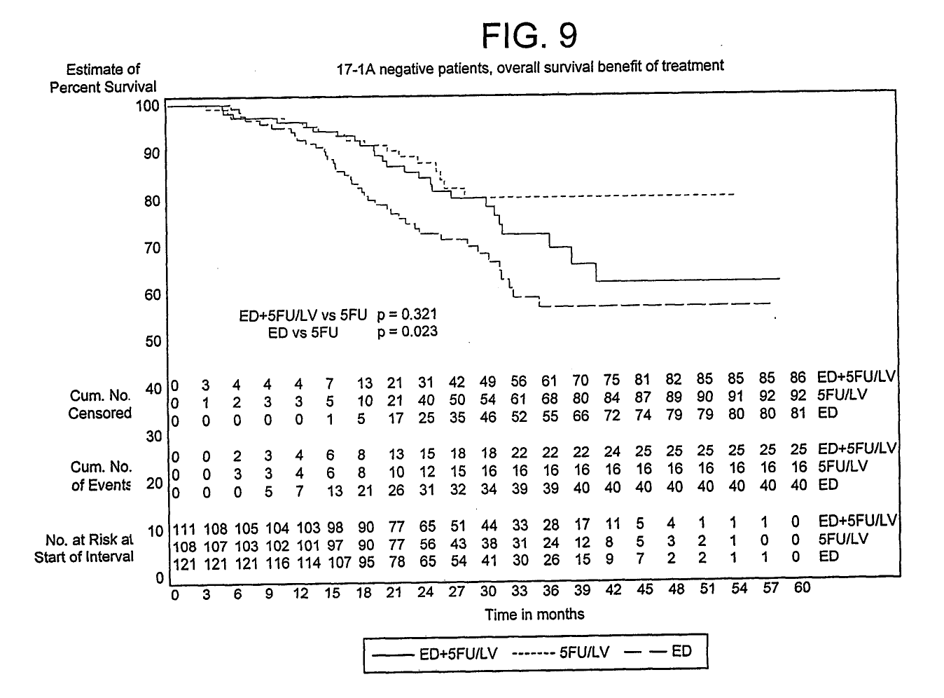

FIG. 9. Overall survival of 17-1A negative patients (2.8 ng/ml 3622 [symbol] antibody dilution)—comparison of the treatment arms.

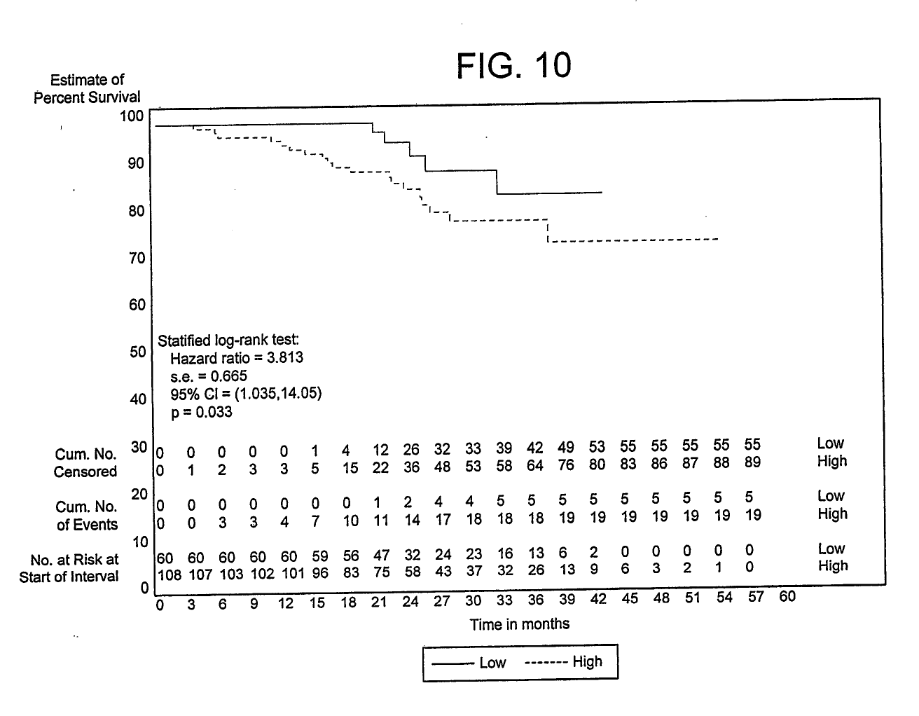

FIG. 10. Overall survival in patients treated with 5FU/LV alone—comparison of patients with TS high and low expression (TS106 antibody at 10 μg/ml). A HR>1 indicates a greater risk of event for patients with TS high tumours.

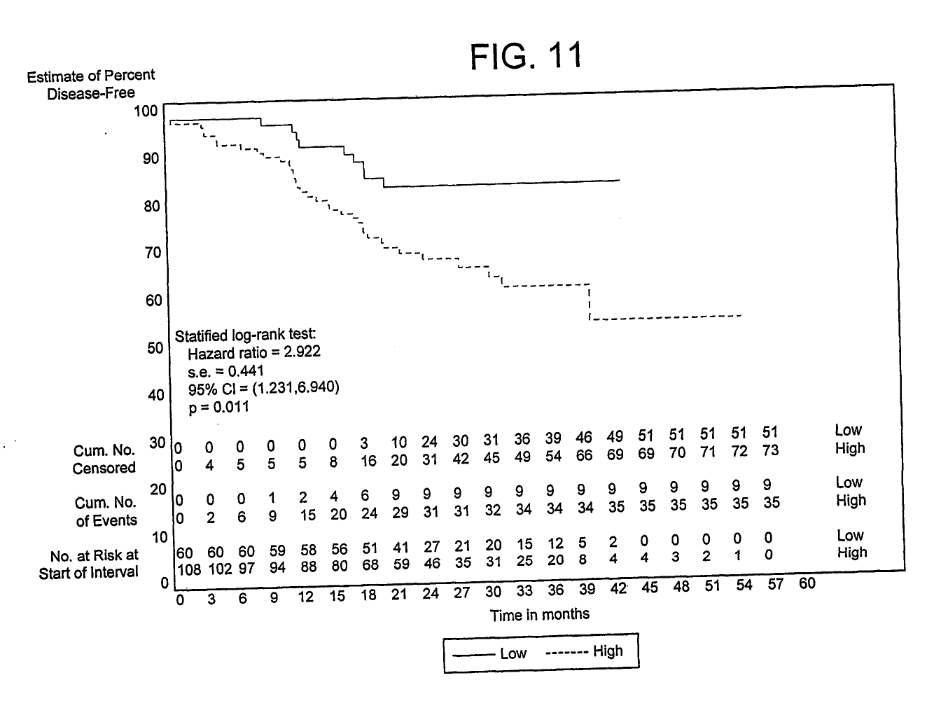

FIG. 11. Disease-free survival in patients treated with 5FU/LV alone—comparison of patients with high and low TS expression (TS106 antibody at 10 μg/ml). A HR>1 indicates a greater risk of event for patients with TS high tumours.

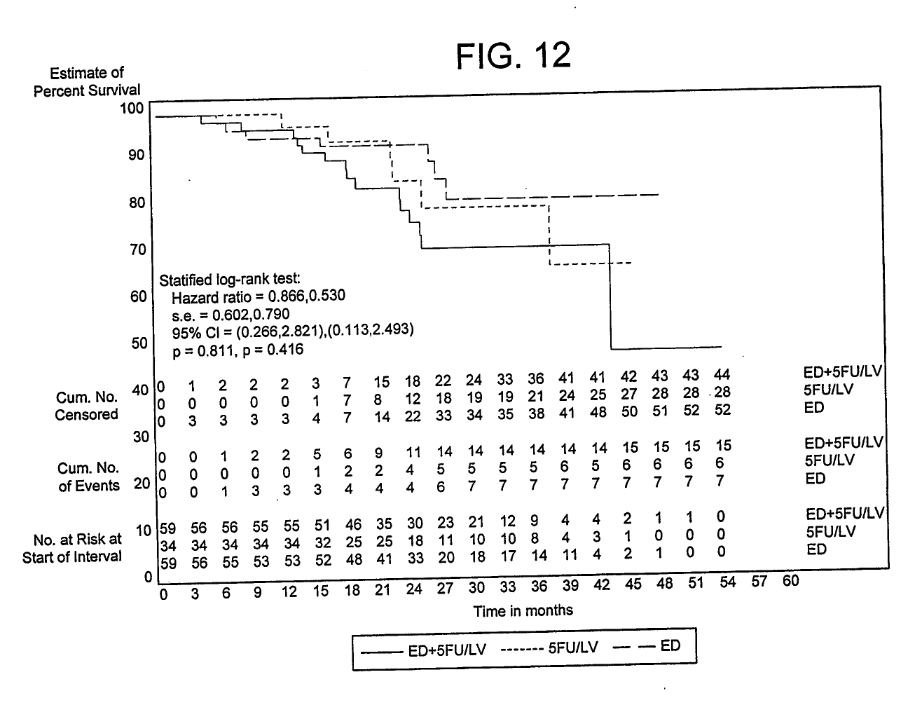

FIG. 12. Overall survival in patients with high TS (10 μg/ml of TS106) and high 17-1A (2.8 ng/ml3622W94). Results of edrecolomab+5FU/LV versus 5FU/LV are presented first followed by results of edrecolomab versus 5FU/LV. A HR<1 indicates a lower risk of event on edrecolomab+5FU/LV or edrecolomab treatment compared to 5FU/LV.

### EXAMPLE 1

### Preparation of FITC-labelled Antibody 3622W94

Antibody 3622W94 (humanised 323/A3 antibody, see Edwards D P, Can.Res, 46, p1306-1317,1986 ) is a high affinity humanised monoclonal antibody directed to the 17-1A antigen. A derivative suitable for utilisation in immunohistochemistry was prepared by attaching a detectable label (fluorescein isothiocyanate, FITC) to the antibody. This reagent is here termed FITC-3622W94. The antibody solution is mixed with FITC in the presence of a bicarbonate buffer for 3 h at room temperature. Using a gel filtration column reacted FITC is separated from free FITC. Bovine albumin is then added to aid stability.

To confirm the specificity of this reagent, samples of frozen human tissue were stained with FITC-3622W94 or another FITC-labelled humanised antibody of irrelevant specificity. This antibody was developed and used for Phase I and II clinical trials, so is very highly purified and specific. The specificity of this detection antibody was demonstrated in a previous immunohistochemistry based study conducted by Wellcome. In this study staining was detected in a series of frozen human tissue samples using fluorescein-labelled 3622W94. The results were compared to staining observed when using an isotype matched fluorescein-labelled whole antibody with irrelevant antigen specificity. The study demonstrated that the 3622W94 antibody bound specifically (via a Fab mechanism) to epithelial cells, including epithelial cells of the large and small intestine. The 17-1A antigen staining was seen to be cytoplasmic/membranous. These findings demonstrate that FITC-3622W94 is a specific and sensitive reagent for the detection of 17-1A antigen expression in human tissue samples.

### EXAMPLE 2

### 17-1A Antigen Immunohistochemistry with Antibody FITC-3622W94

Samples of primary colon tumour tissue were obtained from patients undergoing potentially curative surgery for the treatment of Dukes' stage C colon cancer. These were fixed in formalin and embedded in paraffin according to the standard practice of the hospitals treating the patients.

A typical sample preparation method might be as follows. Add approximately 5 times the volume of the appropriate fixative to the sample and incubate for 4-24 h depending on the optimum for the fixative selected (typically formalin 6-12 h, Bouin's 4 h; ethanol/acid, 24 h). Rinse in three changes of 70% ethanol and place sample in embedding cassette in 70% ethanol. Place the sample and cassette in a tissue processor. The tissue processor dehydrates to 100% alcohol and then infiltrates the tissue with xylene. Xylene saturated tissue is then infiltrated with melted paraffin at 60-70° C. under pressure. Orient the specimen in the cassette, embed in paraffin and cool to a solid block.

Paraffin-embedded fixed colon tumour tissue samples were then analysed for 17-1A antigen expression by immunohistochemistry with FITC-3622W94. Tissue sections of 4 μm were cut onto Superfrost Plus microscope slides and incubated overnight at 37° C. Sections were dewaxed by immersing in warm xylene for 10 minutes and then absolute alcohol ×3. Endogenous peroxidase was blocked using 10 ml of 30% hydrogen peroxide in 400 ml of methanol for 20 min at room temperature. Sections were washed with water before antigen retrieval. To retrieve antigens sections were placed in trypsin solution (200 mg trypsin, 400 ml of distilled water, 8 ml 5% calcium chloride, at 37° C., adjusted to pH 7.8) for 3 min at 37° C. After rinsing in water further antigen retrieval was carried out by microwaving sections in citrate buffer (0.84 g of anhydrous citric acid, 400 ml distilled water, adjusted to pH 6) for 7 min on full power. Sections were rinsed in water then Tris buffered saline (TBS) (6 g Tris-(hydroxymethyl)-Methylamine, 8.5 g sodium chloride, 1 l distilled water, adjusted to pH 7.6). Slides were then loaded onto a Shandon Sequenza immunostaining system. Sections were washed with TBS and non-specific proteins blocked with casein solution (Vector Laboratories cat.no.sp5020) diluted 1:10 with distilled water for 10 min. After washing with TBS sections were incubated at room temperature with the primary antibody (FITC-3622W94) diluted with TBS as appropriate to various concentrations.

Sections were washed with TBS and incubated with a biotinylated antibody against fluorescein (Vector Laboratories, cat.no.BA0601) diluted 1:50 with TBS for 30 min at room temperature. After washing with TBS a complex of horseradish peroxidase (HRP) conjugated streptavidini biotin (Reagents A and B from Dako kit each diluted to 1:100 with TBS, cat.no.K0492) was applied for 30 min at room temperature. Sections were then washed with TBS, removed from the Sequenza system and diaminobenzidine (DAB) was added for 5 min (giving a brown colour in the presence of HRP). Sections were then washed in water and immersed in copper sulphate solution (4 g copper sulphate,7.2 g sodium chloride and 1 l distilled water) for 5 min. After again washing in water nuclei were stained by immersing in Mayers haemotoxylin followed by immersion in acid alcohol and Scott's tap solution. Sections were dehydrated with absolute alcohol and immersed in xylene before mounting with synthetic mounting medium.

### EXAMPLE 3

### Substantiation of Immunohistochemistry Methods

For each run of the immunohistochemistry assay a negative control section was included. It was cut and processed exactly as in example 2, but with the omission of the FITC-3622W94 primary antibody; this was to control for staining due to reagents other than the primary antibody.

For each run of the immunohistochemistry assay a positive control section was included. This was a tissue sample known to express 17-1A antigen and was cut and processed exactly as in example 2; this was to demonstrate that the immunohistochemistry staining process had worked.

On a series of 10 samples, an isotype matched, fluoresceinated, humanised mouse anti-human CD4 antibody was used in place of 3622W94. No specific staining was seen, demonstrating that no characteristics of the 3622W94 primary antibody other than specific binding to the antigen caused staining.

Normal colon samples (n=20) were obtained from patients undergoing resection for colorectal cancer. These samples represent tissue from the resection margins and were judged by a pathologist to be histologically normal, uninvolved in the neoplastic process. These were approximately equally from the left and right side of the bowel. Samples were fixed in formalin and paraffin embedded. These samples were used to optimise the antigen retrieval conditions and then to determine the dilutions of FITC-3622W94 primary antibody to be used for detection of 17-1A antigen on tumour specimens. At dilutions of 1:50,1:100, 1:500; 1:750, 1:1000 and 1:5000 staining, as visually determined, was intense, at dilutions of 1:6500,1:8500 and 1:10,000 there was some loss of intensity, at the 1:100,000 dilution staining was just lost and at 1:500 000 and 1:1 000 000 no staining was visable. The levels of intensity of staining were uniform across all these normal samples.

Three dilutions of FITC-3622W94 were selected for further analysis—1:100, 1:10,000 and 1:100,000. These dilutions correspond approximately to amounts of 2.8 μg/ml, 28.0 ng/ml and 2.8 ng/ml of FITC-3622W94 applied to each tissue section.

### EXAMPLE 4

### Treatment of Patients Afflicted with Dukes' Stage C Colon Cancer

Patients were recruited into a randomised study to evaluate adjuvant therapy of stage III colon cancer. 2761 patients were enrolled through 247 centres in 27 countries. Due to the large numbers of patients involved and the wide geopgraphical spread this patient group is broadly representative of stage III colon cancer patients in the population as a whole.

Randomisation took place between 7 and 42 days post surgery to one of three treatment arms outlined below.
1. Edrecolomab plus 5-fluorouracil (5FU) and leucovorin (LV). The first dose of edrecolomab (500 mg) was administered 7 to 42 days post surgery. The second dose of edrecolomab (100 mg) was administered prior to the first dose of 5FU/LV. A further 3 doses of edrecolomab (100 mg each) were delivered at 4 week intervals. The first cycle of 5FU/LV began at least 14 days after the first dose of edrecolomab and up to 56 days post surgery. It consisted of 20 mg/m² of leucovorin followed by 425 mg/m² of 5FU. 5FU and LV were administered daily for 5 days. The 2^{nd} and 3^{rd} cycle of 5FU/LV were given at 4 weekly intervals and the 4^{th}, 5^{th} and 6^{th} cycles were given at 5 weekly intervals. The second, third and fourth doses of Edrecolomab and the first three cycles of 5-FU/LV were given on the same day. The fifth infusion of Edrecolomab was given one week before the fourth cycle of 5FU/LV.
2. 5FU/LV. The first cycle of 5FU/LV began 7 to 42 days post surgery and consisted of 20 mg/m² of leucovorin followed by 425 mg/m² of 5FU. 5FU and LV were administered daily for 5 days. The 2^{nd} and 3^{rd} cycle were given at 4 weekly intervals and the 4^{th}, 5^{th} and 6^{th} cycles were given at 5 weekly intervals.
3. Edrecolomab. The first dose of edrecolomab (500 mg) was administered 7 to 42 days post surgery. A further 4 doses of edrecolomab (100 mg each) were delivered at 4 week intervals.

We chose to analyse tumour samples from patients enrolled in the study described above. In this instance the patients were all stage III/Dukes C colon cancer and underwent potentially curative resection of a primary tumour. These patients are therefore a good example of patients with minimal residual disease and therefore potentially suitable for treatment with an anti-17-1A targeted therapy. Patients with other diease stages e.g. Dukes stage B or D are additional groups who may be considered to have or be at risk of, minimal residual disease and can potentially benefir from the present invention.

### EXAMPLE 5

### Immunohistochemistry of Tumour Samples for 17-1A Antigen Expression

Due to the large sample group size and the wide geographical spread we believe these cases to be broadly representative of the stage III/Dukes C colon cancer population in general. Amongst this group the mean age was 61.6 years and 50.1% were male.

Tumour samples were obtained from 609 patients for immunohistochemical analysis of 17-1A antigen expression level. Each sample was separately stained with FITC-3622W94 at dilutions of 1:100, 1:10,000 and 1:100,000. Processed tissue sections were scored by a pathologist for intensity and distribution of 17-1A antigen expression according to the following scheme.

| | |
|---|---|
| For intensity | 0 - no staining |
| | 1 - weak staining |
| | 2 - moderate staining |
| | 3 - strong staining |
| For distribution | 0 - no staining |
| | 1 - <10% of cells staining |
| | 2 - 10-69% of cells staining |
| | 3 - ≧70% of cells staining |

The intensity and distribution scores were also combined to create a total score in the range 0-6.

Positive controls run with each batch of slides showed that the staining process had been successful. Negative controls run with each batch of slides showed that staining due to reagents other than the primary antibody had not occurred. The isotype matched fluoresceinated humanised mouse anti-human CD4 antibody controls demonstrated that staining was specific for 17-1A antigen.

It was possible to analyse all samples included in the study. No difference in staining pattern was observed between normal and tumour tissue—all gave homogeneous membranous and cytoplasmic staining. Where there was any staining (intensity ≧1) all cases (except 3) scored 3 for distribution.. Using the 1:100 dilution of the antibody, >99% of tumours were positive for 17-1A expression (intensity score >1), therefore it appears that virtually all tumours express 17-1A antigen to some degree. At an antibody dilution of 1:100,000 the preliminary study showed that staining on normal colonic mucosa was just lost. Therefore, by implication, 44% of the tumours in this study express 17-1A antigen at a level higher than that in normal tissue. A summary of 17-1A antigen detection is shown in Table 1.

TABLE 1
Summary of 17-1A detection

| | Immunohistochemistry | | | | | Positive |
|---|---|---|---|---|---|---|
| FITC- | scores | | | Samples | | staining |
| 3622W94 | In- | Distri- | | | % of | (intensity |
| Dilution | tensity | bution | Combined | Number | total | ≧1) |
| 1:100 | 0 | 0 | 0 | 5 | 0.8% | 99.2% |
| | 1 | 2 | 3 | 1 | 0.2% | |
| | 1 | 3 | 4 | 11 | 1.8% | |
| | 2 | 3 | 5 | 87 | 14.3% | |
| | 3 | 3 | 6 | 505 | 82.9% | |
| 1:10,000 | 0 | 0 | 0 | 64 | 10.5% | 89.5% |
| | 1 | 2 | 3 | 2 | 0.3% | |
| | 1 | 3 | 4 | 354 | 58.1% | |
| | 2 | 3 | 5 | 163 | 26.8% | |
| | 3 | 3 | 6 | 26 | 4.3% | |
| 1:100,000 | 0 | 0 | 0 | 340 | 55.8% | 44.2% |
| | 1 | 3 | 4 | 251 | 41.2% | |
| | 2 | 3 | 5 | 18 | 3.0% | |

### EXAMPLE 6

### Statistical Analysis of 17-1A Antigen Expression Levels

A series of statistical analyses were planned to correlate the results of immunohistochemistry for 17-1A antigen with clinical outcome following treatment with edrecolomab and/or 5FU/LV.

As the distribution of 17-1A antigen was found to be homogeneous statistical analyses were conducted only using the intensity scores. Comparisons were made between patients with tumours positive for 17-1A antigen, i.e. intensity score of 1, 2 or 3 and patients with tumours negative for 17-1A antigen, i.e. intensity score of 0. This comparison was specified prior to analysis as being the comparison of interest, thus limiting the number of statistical tests conducted on the data. This serves to control for multiplicity and reduces the chance of false conclusions.

The preliminary data suggest that there is no advantage in carrying out statistical tests on the data arising from the 1:100 antibody dilution as 99.2% of the patients (604/609) tested positive for 17-1A antigen. Statistical analyses were therefore conducted only on data resulting from the 1:10,000 and 1:100,000 antibody dilutions. It should be noted that the group of patients negative for 17-1A antigen expression at the 1:10,000 dilution was small (n=64/609).

It will be understood throughout the following discussion that the term “17-1A antigen positive” is used to mean a tumour which has detectable staining for 17-1A antigen using the specified concentration of FITC-3622W94 for immunohistochemistry. Similarly “17-1A antigen negative” is used to describe a tumour which lacks detectable staining for 17-1A antigen using the specified concentration of FITC-3622W94 for immunohistochemistry. As the results obtained using the 1:100 dilution of antibody show, virtually all (at least 99.2%) tumours in this study express the 17-1A antigen.

The plan for statistical analyses was determined following examination of the initial 17-1A antigen detection data. This allowed only potentially useful analyses to be carried out and by reducing the number of comparisons reduced the likelihood of falsely significant results.

Analyses focused primarily on the overall survival endpoint, although they were repeated on the secondary disease-free survival endpoint. Plots of Kaplan-Meier survival estimates and hazard ratios (HRs), with corresponding 95% confidence intervals (CIs), were used to compare groups. A stratified log rank test was used to determine whether there was a significant difference in survival for those patients whose primary tumour was positive for 17-1A antigen compared to those patients whose primary tumour was negative for 17-1A antigen. As with the main clinical study analysis, the test was stratified by geographical location and nodal status and conducted on the intent-to-treat population. A comparison was made using each individual randomised treatment arm, to determine whether 17-1A antigen positivity is predictive of survival for the individual treatment schedules. Comparisons were also made between benefits from each treatment in the 17-1A antigen positive and negative patients separately. No adjustment was made for multiple comparisons.

### EXAMPLE 7

### 17-1A Antigen is not a General Prognostic Marker for Colon Cancer

To determine whether 17-1A antigen expression levels act as a general prognostic marker for colon cancer, the survival outcome of 17-1A antigen positive and 17-1A antigen negative patients was compared amongst those patients not treated with a 17-1A targeting therapy. This analysis was performed using data from both the 1:10,000 and 1:100,000 dilutions of FITC-3622W94. No significant relationship was found between 17-1A antigen status and overall or disease-free survival following 5FU/LV treatment, indicating that 17-1A antigen expression levels do not act as a general prognostic marker for colon cancer.

### EXAMPLE 8

### Analysis Using the 1:10,000 Dilution of FITC-3622W94

Immunohistochemical data obtained using 1:10,000 dilution of FITC-3622W94 were examined in two ways. Initially patients were defined as negative (intensity score 0) or positive (intensity score 1, 2 or 3) for 17-1A antigen expression. This divided the population into 89% positive and 11% negative patients. No significant differences in overall or disease free survival were seen between patients positive or negative for 17-1A antigen in any of the treatment arms.

These results show that grouping patients into negative and positive for 17-1A antigen using this dilution of the detection antibody does not discriminate amongst patients with differing benefits from edrecolomab.

Additionally the data was split to compare low expressors of 17-1A antigen (intensity score 0,1) to high expressors of 17-1A antigen (intensity score 2,3). Again this was to find whether using the 1:10,000 dilution of the detection antibody the patients could be grouped in a such a way that a cut-off point at which level of expression of 17-1A is associated with benefit from edrecolomab was identified. Dividing the population into groups by this definition did not distinguish between those benefiting and not benefiting from edrecolomab.

### Example 9

### Analysis Using the 1:100,000 Dilution of FITC-3622W94

Using the immunohistochemistry results produced with a dilution of 1:100,000 FITC-3622W94, patients were defined as negative if 17-1A antigen expression was below the level of detection (intensity score 0) or positive (intensity score 1 or 2) for 17-1A expression. This divided the population into 44% positive and 56% negative patients and outcome for the two groups was compared.

Those patients with tumours positive for 17-1A antigen expression using these immunohistochemistry conditions had a significantly improved overall survival over patients with tumours negative for 17-1A expression if treated with edrecolomab alone (Hazard ratio (HR)=0.42 (95% confidence interval (Cl) 0.21, 0.84) p=0.01 1) (FIG. 1). This difference was not seen in the patients treated with 5FU/LV (HR=0.75 (95% Cl 0.30, 1.90) p=0.547) (FIG. 2) or in those treated with 5FU/LV +edrecolomab (HR=1.10 (95% Cl 0.60, 2.01) p=0.768) (FIG. 3).

The effect of 17-1A antigen status on disease-free survival was also considered. The trend for improved outcome if positive for 17-1A expression and treated with edrecolomab alone, seen for overall survival, was weak for disease-free survival (HR=0.78 (95% Cl 0.50, 1.24) p=0.297) (FIG. 4). As was seen for overall survival there was no improved disease free survival for patients positive for 17-1A, if treated with 5FU/LV or 5FU/LV+edrecolomab (FIGS. 5 and 6).

Riethmuller et al. (1994) investigated edrecolomab treatment in Dukes' stage C colon cancer patients and concluded that edrecolomab gave significant protection from distant recurrence but not from local recurrence. When time to distant metastasis (as a first event) was considered those patients treated with edrecolomab had a significant advantage over those given no adjuvant treatment (p=0.001). This was not the case for time to local recurrence, where there was no difference between patients receiving edrecolomab and patients receiving no adjuvant treatment (p=0.740). As treatment with edrecolomab is expected to be most effective against dispersed cells and less effective against larger masses of tumour cells, it follows that the value of 17-1A antigen expression in predicting improvement in disease-free survival would be expected to be greater in the patients with distant metastases as a first event compared to those with local recurrence.

To examine this in the current data set an additional analysis was carried out whereby disease-free survival was plotted for patients treated with edrecolomab alone, but including only those whose first recurrence was a distant metastasis. The weak trend towards improved disease-free survival for 17-1A antigen positive patients (p=0.297) did, as expected, become stronger if only those patients recurring with distant metastases as a first event were included in the analysis (HR=0.65 (95% Cl 0.39,1.08) p=0.093) (FIG. 7).

When considering only patients positive for 17-1A antigen, the overall survival of patients treated with edrecolomab alone was not significantly different to those treated with 5FU/LV (p=0.734); however the HR weakly favoured 5FU/LV (HR=1.182 (95% Cl 0.45, 3.10)) (FIG. 8). Amongst those patients with tumours negative for 17-1A antigen expression overall survival is significantly poorer if treated with edrecolomab alone than 5FU/LV alone (p=0.023) (FIG. 9).

Patient characteristics were compared between the 17-1A antigen positive and negative groups (Table 2). This was to find whether the significant overall survival difference for 17-1A antigen positive and negative patients, treated with edrecolomab alone, was contributed to by other factors. The patient characteristics appeared well balanced in the edrecolomab treated group, between the 17-1A antigen positive and negative subsets, except for nodal status and mucin status. Nodal status had been adjusted for in the initial analyses, along with geographical location, but mucin status had not. Therefore the log rank test was repeated adjusting additionally for mucin status and the results were found not to be affected.

The data indicates that immunohistochemical analysis of colon tumours for expression of the 17-1A antigen can be used to identify a group of patients who may derive most benefit from treatment with edrecolomab (the “17-1A antigen positive” group identified using 1:100,000 FITC-3622W94). Similarly, the same test can be used to delineate a group of patients who may benefit less from edrecolomab treatment (the “17-1A antigen negative” group), who may preferably be treated with alternative therapies such as 5FU/LV.

### EXAMPLE 10

### Further Methods for Detection of 17-1A Antigen Expression Levels

Although the present invention is described using FITC-3622W94 as a suitable reagent for assessing levels of 17-1A antigen, it will be appreciated that other antibodies directed to this antigen may also be readily used. Examples of such antibodies include GA733 (Herlyn D. j.immunological methods, 73,157-167,1984) and 323/A3 (Edwards D et al, Can.Res 46,1306-1317,1986). To use such reagents in the practise of the invention, a comparative study is conducted.

Because such antibodies will differ from FITC-3622W94 in parameters such as affinity for the 17-1A antigen, a calibration procedure must be performed in order to ensure that appropriate immunohistochemical staining is obtained, and the desired patient population correctly identified. If the alternative antibody is not labelled with FITC, but bears another detectable moiety, appropriate modifications will be made to the methodology to enable its detection. These are well known to those skilled in the art.

Similarly, to those skilled in the art of immunohistochemistry, it will be apparent that different batches of the same detection antibody (eg FITC-3622W94) may have different potencies in use, and that each batch must be appropriately calibrated to correctly identify the desired patient population.

Such calibration procedures can be accomplished in a number of ways, but the aim is always the same—to identify the conditions of detection and concentration of the alternative antibody which will segregate the colon cancer population into similar groups identified by FITC-3622W94 in the study outlined in example 9. Two examples of calibration procedures are given below.

In one calibration method, the samples of colon cancer tissue to be investigated are stained with the alternative anti-17-1A antibody. The cases are then ranked according to intensity of staining and the 44% with the greatest intensity of staining are considered as 17-1A high expressors and those remaining (56%) with lesser intensity staining are low expressors. These groupings may then be used to determine the suitability of patients for treatment with agents that target 17-1A antigen such as edrecolomab. That is to say, those cases in the top 44% represent the preferred group for treatment with a 17-1A antigen-targeting therapy, such as edrecolomab. After using the above disclosed method to correctly calibrate the detection antibody to be used, the intensity of staining obtained in those cases is used as a reference expression level. Those tumours close to the cut-off point between the high and low expressing groups (eg the tumours with the lowest staining amongst the high expressing group, and the tumours with the highest staining of the low expressing group) are now used as a reference expression level. This reference is that which individual tumour samples are then compared against in order to determine suitability of an individual for treatment with a 17-1A antigen-targeting therapy.

It is not necessary to achieve a precise 44%:56% split in the sample series in order to practice the invention. Staining procedures that result in a similar, but not identical, segregation may also be used. This is clear from the following example;

41 patients were selected. 17 were treated with surgery and edrecolomab and enjoyed an encouraging clinical course, showing no local or distant recurrence of their disease for up to 2 years post-surgery. 24 patients, treated identically, had a worse outcome, with no local recurrence, but with disease recurring at a site distant from the primary lesion between 6 and 18 months after resection. The primary tumours resected from these patients were analysed for their 17-1A expression levels. Patients whose tumours had high 17-1A expression (14/41, 34%) had an extended disease-free survival time (778 days) compared to the 66% (27/41) with the lowest 17-1A expression (630 days). This shows that ranking the 17-1A expression in such a way as to split the population in the ratio 34%:66% is sufficient to select a favorable population for treatment. Similarly, ratios up to 54%:46% may also be used.

A second alternative calibration method may also be employed. Again, the objective is to identify the correct conditions of detection and concentration of an alternative antibody which will segregate the colon or colorectal cancer population into similar groups identified by FITC-3622W94 in the study outlined in example 9. A series of normal colonic mucosa samples is stained (e.g. samples from 30 different individuals) with a range of concentrations of the alternative antibody. The concentration at which approximately 80% of samples of normal tissue (preferably of the same tissue type as the tumour being assessed) and greater than or equal to 34% of the tumour samples are positive is then selected for use to identify high and low 17-1A expressors.

For example a batch of 3622W94 FITC labelled in a separate reaction to the batch used in the examples described above was required for use. Clearly as it was labelled in a separate reaction to the batch of antibody used in the examples described above it needed to be recalibrated. It was assessed at a concentration of approximately 16.5 ng/ml where 10/10 normal samples and approximately 7/10 tumour samples were positive. This did not fulfill the criteria above and was not selected. It was assessed at a concentration of approximately 2.8 ng/ml where approximately 3/40 normal samples and approximately 5/40 tumour samples were positive. This did not fulfill the criteria above and was not selected. A third concentration of approximately 11.2 ng/ml was then assessed. In this case approximately 10/50 normal samples and 29/50 tumour samples were positive. Therefore the selection criteria outlined above were fulfilled and this concentration selected for immunohistochemistry in order to identify patients particularly suitable for treatment with 17-1A antigen-targeting therapies.

By following this procedure an appropriate concentration of the alternative or new batch of antibody is determined. This concentration will then be used to assess the suitability of individual patients for treatment with an anti-17-1A targeting therapy. Using this method the concentration of alternative antibody determined will stain positively those cases most suitable for treatment with a 17-1A targeting therapy. In order to identify this positivity a reference standard may be used, for example a tumour known to stain negatively at this concentration.

The accuracy of this calibration process was demonstrated by repeating staining of 50 cases from the original group of 609 using the recalibrated new batch of FITC-labelled 3622W94 at a concentration of approximately 11.2 ng/ml. Using this recalibrated antibody concentration 44/50 (88%) cases gave concordant staining results compared with those obtained when using the batch of FITC-3622W94 used in the examples above at approximately 2.8 ng/ml, a result well within expected experimental error for immunohistochemistry. This recalibrated concentration of the new antibody batch is then used to stain the samples of colon cancer tissue to be investigated. The tumour samples which are positive for 17-1A are high expressors. As demonstrated in example 9 those patients with a high expression gain a greater benefit from the 17-1A targeting antibody therapy edrecolomab. These groupings may then be used to determine the suitability of patients for treatment with agents that target 17-1A antigen such as Edrecolomab or MT201.

It will be understood that the purpose of these calibration methods is to adjust the immunohistochemistry conditions so as to identify a proportion of the general cancer population who are particularly suitable for 17-1A antigen-targeting therapy. Clearly though, there may be circumstances where it is desired not to treat the entire patient group so identified, but to seek a subset of the group who may be even more suitable for such therapy. These methods may also be applied to calibrate the 17-1A detection methods in such a way as to enable definition of such a smaller group. For example, in the first calibration method (supra), cases are ranked on their expression level as described, but instead of selecting for treatment the 44% with the greatest intensity of staining, a more restrictive cut-off is chosen, perhaps treating only the most intense 20%, or 10%, or 5% or even 2%. Without wishing to be bound by theory, we propose that the more stringent the treatment selection hurdle chosen in this way, the progressively more suitable the patients may be for 17-1A antigen-targeting therapy.

### EXAMPLE 11

### Combination of 17-1A Expression Analysis With Other Prognostics

Since both edrecolomab and 5FU/LV treatment of Dukes' stage C colon and colorectal cancer patients has been shown to give clinical benefit, it would be highly desirable to be able to combine these treatments in the most effective manner. Similarly, there are other chemotherapies for the treatment of colorectal cancer with which it would be useful to combine 17-1A targeting therapies such as edrecolomab. Examples include—raltitrexed, Oxaliplatin, Irinotecan/CPT11, eniluracil (with 5FU ±leucovorin), UFT, Capecitabine, mitomycin, Herceptin.

Combinations including such therapies may also be directed or determined by prognostic markers for degree of benefit from these treatments.

The cytotoxic mechanism of action of 5FU falls into two main parts: incorporation of fluorinated ribonucleotides into RNA and inhibition of thymidylate synthase (TS) with subsequent effects on DNA synthesis and repair.

The TS enzyme, which is inhibited by metabolites of 5FU, is a crucial enzyme in nucleotide metabolism as its role in deoxythymidine triphosphate (dTTP) synthesis cannot be compensated for by another route. Normally TS binds to dUMP and then to a folate cofactor, CH₂FH₄. TS catalyses the conversion of dUMP to deoxythymidine monophosphate (dTMP) which is further phosphorylated to dTTP for incorporation into DNA (Danenberg & Danenberg, 1978).

After metabolism of 5FU to FdUMP it competes with dUMP for the catalytic site on TS. Once FdUMP is bound to TS, binding of the folate cofactor occurs and the enzyme, the folate and the FdUMP become covalently bound in a ternary complex (Santi et al. 1974). By inhibiting TS, 5FU prevents the conversion of dUMP to dTMP and the result is thymine depletion, which can lead to cell death. It has been suggested that the cytotoxic action of 5FU can be shifted from RNA to DNA based effects by the co-administration of LV which is metabolised to CH₂FH₄ to provide a higher level of the folate cofactor. This increases the stability of the ternary complex and hence the inhibition of TS.

The value of TS as a predictive marker is important to investigate. It may allow the discrimination of patients into those who would benefit most from 5FU based therapy and those who should be considered for alternative or additional therapies.

A preliminary study of 9 disseminated colorectal tumours, found that those responding to 5FU/LV therapy had a lower mean TS protein level and mRNA level in pre-treatment biopsies than those not responding (p<0.01). This study also found that immunohistochemical staining intensity of TS correlated closely with protein expression measured by Western blot and mRNA level assessed by rtPCR (Johnston et al. 1995).

Two studies of patients with advanced colorectal cancer went on to confirm these findings. Leichman et al. (1997) measured TS mRNA in pretreatment biopsies from 42 patients using β-actin mRNA level as an internal control. The patients gained varying benefit from 5FU/LV and length of survival was significantly higher in the patients with a low TS to β-actin ratio compared to those with a high ratio (p=0.02). Lenz et al. (1998) used a similar method to assess expression of TS mRNA in 36 patients treated with 5FU/LV. Patients with low pretreatment TS levels had a significantly greater response to therapy (p=0.003) and survived for significantly longer (p=0.002). Not all of the patients with low TS levels responded and so other factors are likely to be involved. Immunohistochemistry based studies have identified similar relationships (Aschele et al. 1999; Paradiso et al. 2000).

Earlier stage disease has also been investigated. However in this case TS level is measured in a different tumour (primary) to that being treated with the 5FU (metastases or recurrent disease). There is reason to believe that this may be important as TS levels have been found to be significantly higher in primary tumours than in hepatic metastases (Chazal et al. 1997). However, TS level was measured by immunohistochemistry in the primary tumours of 100 Dukes' stage C colon cancer patients treated with 5FU-based chemotherapy; it was found that the majority suffering a relapse had TS overexpressing tumours and the majority who remained disease free had TS negative tumours (Cascinu et al. 2001). While this study appeared to fit with the results from the advanced stage disease studies, a second investigation of 134 early stage colorectal disease found no relationship between TS expression, measured by immunohistochemistry in the primary tumour, and benefit from 5FU or raltitrexed (Findlay et al. 1997). However in this study only distribution of TS was assessed and not intensity of staining.

The relationship between TS level and benefit from 5FU may be complicated by other factors. For example Van der Wilt et al. showed in in vivo colon tumour models that levels of TS increased as a result of treatment with 5FU (van der Wilt et al. 1992). Chu et al. (1991) found in vitro evidence demonstrating that the TS enzyme regulates the translation of its own mRNA, so when substrates or inhibitors of the enzyme are present, inhibition of translation is lifted. Resistance to 5FU could also be due to mutations of TS resulting in interference of binding to the folate or to FdUMP.

From this survey of the literature it is apparent that the link between TS level and outcome after 5FU based therapy is clear for advanced disease, but not resolved for earlier stage disease.

### EXAMPLE 12

### Method for Staining for Thymidylate Synthase Expression Levels

Sections from the same formalin-fixed paraffin-embedded tissue samples described above analysed by immunohistochemistry for level of thymidylate synthase. Sections of 4 μm were cut onto Superfrost Plus microscope slides and incubated overnight at 37° C. Sections were dewaxed by immersing in warm xylene for 10 minutes and then absolute alcohol ×3. Endogenous peroxidase was blocked using 10 ml of 30% hydrogen peroxide in 400 ml of methanol for 20 min at room temperature. Sections were washed with water before antigen retrieval. To retrieve antigens sections were placed in Vector antigen retrieval solution (Vector, cat.no.H330) in a Prestige pressure cooker at full pressure for 3 min. Sections were rinsed in water then tris buffered saline (TBS) (6 g Tris-(hydroxymethyl)-Methylamine, 8.5 g sodium chloride, 1 l distilled water, adjusted to pH 7.6). Slides were then loaded onto a Shandon Sequenza immunostaining system. Sections were washed with TBS and non-specific proteins blocked with casein solution (Vector Laboratories, Cat.No.Sp5020) for 10 min. After washing with TBS sections were incubated with Avidin solution and Biotin solution (Vector Cat.No.SP2001) for 15 min each to block endogenous biotin. After washing again with TBS the primary antibody (TS106, Chemicon) was added diluted to 1:100 (10 μg/ml) or 1:5000 (200 ng/ml) with casein solution and incubated overnight at 4° C. Sections were washed with TBS and incubated with the secondary antibody (biotinylated anti mouse/rabbit immunoglobulins (Dako, K0492)) diluted appropriately with TBS for 30 min at room temperature. After washing with TBS a complex of streptavidine and biotinylated horseradish peroxidase (HRP) (Dako, K0492) was applied for 30 min at room temperature. Sections were then washed with TBS, removed from the Sequenza system and diaminobenzidine (DAB) was added for 2 min (giving a brown colour in the presence of HRP). Sections were then washed in water and immersed in copper sulphate solution (4 g copper sulphate,7.2 g sodium chloride and 1 l distilled water) for 5 min. After again washing in water nuclei were stained by immersing in Mayers haematoxylin. Sections were dehydrated with absolute alcohol and immersed in xylene before mounting with synthetic mounting medium.

### EXAMPLE 13

### Immunohistochemistry of Tumour Samples for TS Expression

Tumour samples were obtained from 609 patients for immunohistochemical analysis of TS expression level. Each sample was separately stained with antibody TS106 at dilutions of 1:100 or 1:5,000. Processed tissue sections were scored by a pathologist for intensity and distribution of TS expression according to the following scheme.

| | |
|---|---|
| For intensity | 0 - no staining |
| | 1 - weak staining |
| | 2 - moderate staining |
| | 3 - strong staining |
| For distribution | 0 - no staining |
| | 1 - <10% of cells staining |
| | 2 - 10-69% of cells staining |
| | 3 - ≧70% of cells staining |

The intensity and distribution scores were also combined to create a total score in the range 0-6.

### EXAMPLE 14

### Statistical Analysis of TS Expression Levels

A statistical analysis was conducted of the relationship between clinical outcome and TS expression levels as determined by immunohistochemistry using antibody TS106 at a dilution of 1:100. Outcome of patients with tumours with a low level of TS expression (total score 0-3, 39.5% of patients) was compared to a high level of expression (total score 4-6, 60.5% of patients). These groups will be termed “TS low” and “TS high” respectively throughout the following discussion.

To determine whether TS expression levels act as a general prognostic marker for colon cancer, the survival outcome of TS low and TS high patients was compared in patients not treated with a TS targeting therapy. No significant association was found between level of expression of TS and overall survival (HR=0.839, p=0.566) or disease-free survival (HR=0.730, p=0.173) when treated with edrecolomab monotherapy. This suggests that TS expression is not a general prognostic marker for colon cancer.

A high level of expression of TS is associated with a significantly poorer overall survival if treated with 5FU/LV alone (HR=3.813, p=0.033; in this analysis a HR>1 indicates a greater risk of event for high TS expressors). These data are shown in FIG. 10. This is not the case for patients treated with edrecolomab alone (HR=0.839, p=0.566). Similarly, looking at the disease-free survival endpoint, high level of expression of TS is associated with a significantly poorer disease-free survival if treated with 5FU/LV alone (HR=2.922, p=0.011) FIG. 11. This is not the case for patients treated with edrecolomab alone.

Level of TS expression therefore appears to be a predictive marker for benefit from 5FU/LV, but not for treatment with edrecolomab.

Characteristics were balanced between treatment arms and TS status (i.e. low v high), except for mucin status where 25% of high expressors compared to 13% of low expressors had mucinous adenocarcinomas. This difference was sufficient to influence the analysis, and so comparisons of high vs low were repeated adjusting for mucinous status as well as nodal status and geographical region which were originally adjusted for. Therefore any significant results presented are independent of geographical location, nodal status and mucinous status.

### EXAMPLE 15

### Combination of 17-1A Antigen and TS Expression Level Testing

Considering only patients with a high level of expression of TS, i.e. those gaining the least benefit from 5FU/LV. Neither overall survival nor disease-free survival were significantly different for edrecolomab alone treated patients compared to 5FU/LV treated patients. However, there were weak trends towards edrecolomab alone giving a poorer overall survival (HR=1.507, p=0.181; in this analysis, a HR>1 indicates a greater risk of event for patients treated in the experimental arm (edrecolomab alone or edrecolomab+5FU/LV) compared to 5FU/LV alone) and disease-free survival (HR=1.376, p=0.164) than 5FU/LV alone, The survival following edrecolomab treatment is not significantly different to the survival following 5FU/LV in this high TS group. Therefore if the 17-1A antigen positive expressors (who were found to benefit most from edrecolomab monotherapy) were identified amongst this group they would be expected to have a treatment benefit from edrecolomab alone which is at least equivalent if not greater than benefit from 5FU/LV. This was indeed the case as when considering those patients with high TS and high 17-1A status edrecolomab appeared slightly superior to 5FU/LV in terms of overall survival (HR=0.530, p=0.416) and disease-free survival (HR=0.707, p=0.521). See FIG. 12.

Similarly, using the tests for 17-1A and TS expression levels, it is possible to identify a population of patients gaining the greatest benefit from combination therapy (5FU/LV plus edrecolomab) compared to 5FU/LV alone.

Similarly, using the tests for 17-1A and TS expression levels, it is possible to identify a population of patients gaining the least benefit from treatment with either edrecolomab, 5FU/LV or a combination of edrecolomab and 5FU/LV, and so would be good candidates for evaluating alternative therapies.

Similarly, using combinations of other markers together with 17-1A and/or TS, which are demonstrated to have predictive value for degree of benefit from other therapies (such as Her2/neu for benefit from Herceptin), edrecolomab alone or in combination with 5FU/LV can be combined with these other therapies in such a way that patient groups with the greatest potential to benefit are identified. FIG. 1.

References

Aschele, C., Debernardis, D., Casazza, S., Antonelli, G., Tunesi, G., Baldo, C., Lionetto, R., Maley, F. & Sobrero, A. (1999). Immunohistochemical quantitation of thymidylate synthase expression in colorectal cancer metastases predicts for clinical outcome to fluorouracil-based chemotherapy. Journal of Clinical Oncology, 17, 1760-1770.

Cascinu, S., Graziano, F., Valentini, M., Catalano, V., Giordani, P., Staccioli, M. P., Rossi, C., Baldelli, A. M., Grianti, C., Muretto, P. & Catalano, G. (2001). Vascular growth factor expression, S-phase fraction and thymidylate synthase quantitation in node-positive colon cancer: relationships with tumour recurrence and resistance to adjuvant chemotherapy. Annals of Oncology, 12, 239-244.

Chazal, M., Cheradame, S., Formento, J. L., Francoual, M., Formento, P., Etienne, M. C., Francois, E., Richelme, H., Mousseau, M., Letoublon, C., Pezet, D., Cure, H., Seitz, J. F. & Milano, G. (1997). Decreased folylpolyglutamate synthetase activity in tumours resistant to fluorouracil folinic acid treatment: clinical data. Clinical Cancer Research, 3, 553-557.

Chu, E., Koeller, D. M., Casey, J. L., Drake, J. C., Chabner, B. A., Elwood, P. C., Zinn, S. & Allegra, C. J. (1991). Autoregulation of human thymidylate synthase messenger-RNA translation by thymidylate synthase. Proceedings Of The National Academy Of Sciences Of The United States Of America, 88, 8977-8981.

Danenberg, P. V. & Danenberg, K. D. (1978). Effect of 5,10-methylenetetrahydrofolate on the dissociation of 5-fluoro-2′-deoxyuridylate from thymidylate synthetase: Evidence for an ordered mechanism. Biochemistry, 17, 4018-4024.

Dukes, C. E. (1932). The classification of cancer of the rectum. Journal Of Pathology and Bacteriology, 35, 323-332.

Findlay, M. P. N., Cunningham, D., Morgan, G., Clinton, S., Hardcastle, A. & Aherne, G. W. (1997). Lack of correlation between thymidylate synthase levels in primary colorectal tumours and subsequent response to chemotherapy. British Journal Of Cancer, 75, 903-909.

Finlay, I. G. & McArdle, C. S. (1983). Effect of occult hepatic metastases on survival after curative resection for colorectal carcinoma. Gastroenterology, 85, 596-599.

Fisher, E. R. & Turnbull, R. B. (1955). The cytologic demonstration and significance of tumour cells in the mesenteric venous blood in patients with colorectal carcinoma. Surgery, Gynecology and Obstetrics, 100, 102-108.

Gastl, G., Spizzo, G., Obrist, P., Dunser, M. & Mikuz, G. (2000). Ep-CAM overexpression in breast cancer as a predictor of survival. The Lancet. 356, 1981-1982.

Johnston, P. G., Lenz, H. J., Leichman, C. G., Danenberg, K. D., Allegra, C. J., Danenberg, P. V. & Leichman, L. (1995). Thymidylate synthase gene and protein expression correlate and are associated with response to 5-fluorouracil in human colorectal and gastric tumours. Cancer Research, 55, 1407-1412.

Leichman, C. G., Lenz, H. J., Leichman, L., Danenberg, K., Baranda, J., Groshen, S., Boswell, W., Metzger, R., Tan, M. & Danenberg, P. V. (1997). Quantitation of intratumoral thymidylate synthase expression predicts for disseminated colorectal cancer response and resistance to protracted-infusion fluorouracil and weekly leucovorin. Journal Of Clinical Oncology, 15, 3223-3229.

Lenz, H. J., Hayashi, K., Salonga, D., Danenberg, K. D., Danenberg, P. V., Metzger, R., Banerjee, D., Bertino, J. R., Groshen, S., Leichman, L. P. & Leichman, C. G. (1998b). p53 point mutations and thymidylate synthase messenger RNA levels in disseminated colorectal cancer: an analysis of response and survival. Clinical Cancer Research, 4, 1243-1250.

Moertel, C. G., Fleming, T. R., MacDonald, J. S., Haller, D. G., Laurie, J. A., Tangen, C. M., Ungerleider, J. S., Emerson, W. A., Tormey, D. C., Glick, J. H., Veeder, M. H. & Mailliard, J. A. (1995). Fluorouracil plus levamisole as effective adjuvant therapy after resection of stage-III colon-carcinoma—a final report. Annals Of Internal Medicine, 122, 321-326.

Paradiso, A., Simone, G., Petroni, S., Leone, B., Vallejo, C., Lacava, J., Romero, A., Machiavelli, M., De Lena M., Allegra, C. J. & Johnston, P. G. (2000). Thymidylate synthase and p53 primary tumour expression as predictive factors for advanced colorectal cancer patients. British Journal of Cancer, 82, 560-567.

Parkin, D. M., Muir, C. S., Whelan, S. L., Gao, Y. T., Ferlay, J. & Powell, J. (1992). Cancer Incidence in Five Continents, 6, 304, 305, 456,457, 772, 773.

Piyathilake, C. J., Frost, A. R., Weiss, H., Manne, U., Heimburger, D. C. & Grizzle, W. E. (2000). The expression of Ep-CAM (17-1A) in squamous cell cancers of the lung. Human Pathology. 31, 482-487.

Poczatek, R. B., Myers, R. B., Manne, U., Oelschlager, D. K., Weiss, H. L., Bostwick, D. G., Grizzle, W. E. (1999). Ep-CAM levels in prostatic adenocarcinoma and prostatic intraepithelial neoplasia. Journal of Urology. 162, 1462-1466.

Riethmuller G., Holz, E., Schlimok, G., Schmiegel, W., Raab, R., Hoffken, K., Gruber, R., Funke, I., Pichimaier, H., Hirche, H., Buggisch, P., Witte, J. & Pichimayr R. (1998). Monoclonal antibody therapy for resected Dukes' C colorectal cancer: seven year outcome of a multicentre randomized trial. J Clin. Oncol., 16, 1788-1794.

Riethmuller G, et al (1994); Randomised trial of monoclonal antibody for adjuvant therapy of resected Dukes+ C. colorectal carcimona, Lancet 343 1177-83.

Santi, D. V., McHenry, C. S. & Sommer, H. (1974). Mechanism of interaction of thymidylate synthetase with 5-fluorodeoxyuridylate. Biochemistry, 13, 471-480.

Turnbull, R. B., Kyle, K., Watson, F. R. & Spratt, J. (1967). Cancer of the colon: the influence of the no-touch isolation technic on survival rates. Annals Of Surgery, 166, 420-427.

van der Wilt, C. L., Pinedo, H. M., Smid, K. & Peters, G. J. (1992). Elevation of thymidylate synthase following 5-fluorouracil treatment is prevented by the addition of leucovorin in murine colon tumors. Cancer Research, 52, 4922-4928.

1. A method for determining the suitability of a human patient afflicted with colorectal cancer for treatment with a therapeutic agent that specifically binds the 17-1A antigen which method comprises the steps of;
(a) obtaining an ex vivo sample of the cancerous colorectal cells from said patient;
(b) assessing the expression of 17-1A by said cells;
(c) comparing the expression of step (b) with a reference expression level;
(d) determining whether the comparison made in step (c) is indicative of said cells over expressing 17-1A wherein over expression indicates said patient is suitable for treatment with said therapeutic agent.

2. The method of claim 1 wherein the expression of 17-1A antigen is assessed by measuring the amount of binding of a 17-1A antigen specific antibody to said cells under conditions favourable to such binding.

3. The method of claim 2 wherein said 17-1A antigen specific antibody has coupled thereto a detectable moiety.

4. The method of claim 3 wherein said moiety is selected from the group consisting of; radiolabel, fluorescent label, phosphorescent label, colormetric enzyme.

5. The method of claim 2 wherein the 17-1A antigen specific antibody is 3622W94.

6. The method of claim 5 wherein the concentration of said antibody is at 2.8 ng/ml or thereabout.

7. The method of claim 1 wherein the 17-1A antigen specific antibody is other than 3622W94.

8. The method of claim 7 wherein said antibody is at a concentration which provides a substantially equivalent degree of binding as that achieved by 3622W94 at a concentration of 2.8 ng/ml under the same conditions favourable to binding.

9. The method of claim 1 wherein said patient identified as suitable in step (d) is administered a therapeutic antibody that specifically binds to the 17-1A antigen.

10. The method of claim 9 wherein said therapeutic antibody is Edrecolomab.

11. A method of treating a human patient afflicted with colorectal cancer, which method comprises;
(a) performing steps (a) to (d) of claim 1 to determine whether said patient is suitable for treatment with an agent that specifically targets the 17-1A antigen-;
(b) administrating to said patient identified as suitable a therapeutically effective amount of said agent.

12. The method of claim 11 wherein said agent is an antibody.

13. The method of claim 12 wherein said antibody is a humanised, chimeric, human or CDR-grafted antibody.

14. The method of claim 12 wherein said antibody is an IgG antibody.

15. The method of claim 11 wherein said antibody is Edrecolomab.

16. A kit of parts for use in the method of claim 1, said kit comprising a 17-1A specific binding antibody having coupled thereto a detectable moiety, and instructions for measuring 17-1A antigen levels expression in an ex vivo sample of cancer cells at a concentration of said antibody suitable to distinguish between samples over-expressing the 17-1A antigen and those that are not.

17. The kit of claim 16 further comprising a therapeutic 17-1A antigen specific binding agent.

18. A method of treating a human patient afflicted with colorectal cancer, that is unresponsive or partially responsive to 5-flurouracil administered cancer therapy, said method comprising:
(a) performing steps (a) to (d) of claim 1 to determine whether said patient is suitable for treatment with an agent that specifically targets the 17-1A antigen;
(b) administrating to said patient identified as suitable a therapeutically effective amount of said agent.

19. A method of treating a human patient afflicted with cancer, that is unresponsive or partially responsive to one or more chemotherapeutic therapeutic agents for the treatment of cancer which method comprises;
(a) performing steps (a) to (d) of claim 1 to determine whether said patient is suitable for treatment with an agent that specifically targets the 17-1A antigen;
(b) administrating to said patient identified as suitable a therapeutically effective amount of said agent that specifically targets the 17-1A antigen.

20. The method of claim 19 wherein said patient is afflicted with a cancer selected from the group consisting of;
colon, colorectal, breast, gastric, oesophageal, prostate, lung, pancreatic and ovarian cancer.

21. A method for determining the suitability of a human patient afflicted with cancer for treatment with a therapeutic agent that specifically binds to the 17-1A antigen expressed by said cancer, which method comprises the steps of;
(a) obtaining an ex vivo sample of the cancerous cells from said patient;
(b) assessing the expression of 17-1A by said cells;
(c) comparing the expression of step (b) with a reference expression level;
(d) determining whether the comparison made in step (c) is indicative of said cells overexpressing 17-1A wherein overexpression indicates said patient is suitable for treatment with said therapeutic agent;
wherein said cancer is selected from the group consisting of; colorectal, breast, gastric, oesophageal, prostate,lung, pancreatic and ovarian cancer.
